(19) United States
(12) Patent Application Publication
(10) Pub. No.: US 2006/0073470 A1
(43) Pub. Date: Apr. 6, 2006

(54) Method of counting microorganisms or cells

(76) Inventors: Naohiro Noda, Kawasaki-shi (JP); Mutsuhisa Hiraoka, Kawasaki-shi (JP); Kazuhito Takahashi, Kawasaki-shi (JP); Koji Maruyama, Ibaraki-shi (JP); Takeshi Saika, Ibaraki-shi (JP); Yasunobu Tanaka, Ibaraki-shi (JP); Masao Nasu, Osaki-shi (JP); Nobuyasu Yamaguchi, Ibaraki-shi (JP)

Correspondence Address: YOUNG & THOMPSON, 745 SOUTH 23RD STREET, 2ND FLOOR, ARLINGTON, VA, 22202, US

(21) Appl. No.: 10/515,994

(22) PCT Filed: May 28, 2003
(86) PCT No.: PCT/JP03/06693

§ 371 Date: Nov. 30, 2004

(30) Foreign Application Priority Data
May 30, 2002 (JP) 2002-156683

(51) Int. Cl. C12Q 1/00; C12Q 1/04

(52) U.S. Cl. 435/4; 435/34

(57) ABSTRACT

A method of counting microorganisms or cells in a sample by labeling the microorganisms or cells with a fluorescent labeling reagent, which comprises contacting the sample containing the microorganisms or cells with an adhesive sheet having an adhesive layer laminated on at least one surface of a substrate to capture the sample; before fluorescent-labeling the microorganisms or cells, obtaining a first fluorescent image of the sample; after fluorescent-labeling the microorganisms or the cells, obtaining a second fluorescent image of the sample, and then determining the difference in the number of luminous points between the first and second images, or determining a differential image between the first and second images and determining the number of luminous points in this differential image, or determining the number of luminous points among the luminous points in the second image located outside non-sensitive regions attached to the individual luminous points in the first image.

### TECHNICAL FIELD

The present invention relates to a method of counting microorganisms or cells in a sample by labeling the microorganisms or the cells with a fluorescent labeling reagent, and obtaining a fluorescent image.

### BACKGROUND ART

Detection of microorganisms in samples, and tissues and cells of animals and plants is an extremely important matter in an industrial view point in order to confirm sterilization and find abnormality of viable cells. For convenience, the following description is mainly based on bacteria.

Under a natural environment, many viable bacteria are difficult to be cultured. The bacteria do not form colonies on a general agar plate medium and often do not grow on a liquid culture medium. Therefore, the bacteria may not be detected by a conventional culture method.

To solve the problem, fluorescein diacetate (FDA), carboxy fluorescein diacetate (CFDA), 5-cyano-2,3-ditolyl tetrazolium chloride (CTC) and the like that are metabolized in the bacteria and exhibit fluorescence are used to detect physiologically active bacteria. Alternatively, diamidinophenyl indol (DAPI) and acridine orange (AO) that bond to DNA are used to label genes, whereby the bacteria are detected.

The FDA and the CFDA are hydrolyzed by enzymes (esterase) within the microorganisms such as bacteria or the cells, and exhibit fluorescence. The CTC is reduced by breathes of the microorganisms or the cells, and exhibit fluorescence. All reagents described above are contacted with the microorganisms or the cells in the sample to be detected in a solution, taken into and reacted with the microorganisms or the cells, and emit fluorescence. The fluorescence detects the cells or the microorganisms such as the bacteria.

However, in the method of detecting the bacteria with fluorescence, when fluorescent contaminants coexist in the sample, the contaminants are false detected, resulting in a counting error.

In order to overcome the problem, a patent document 1 described later discloses “a method of detecting a viable cell, comprising the steps of (a) dyeing a medium with a fluorescent enzyme substrate to record a fluorescent image, (b) irradiating light and quenching the medium dyed to record a fluorescent image, and (c) taking a differential image between the fluorescent images obtained in the steps (a) and (b).”

In brief, according to the patent document 1, it is noted that the bacteria labeled with a fluorescent reagent be more easily discolored than fluorescent contaminants originally contained in the sample, and effects of the fluorescent contaminants are eliminated using the above-described steps.

However, the patent document 1 has also the following problems:

Although the microorganisms or the cells labeled with the fluorescent reagent are more easily discolored than the fluorescent contaminants contained in the sample, fluorescent properties of the contaminants cannot be controlled. Accordingly, only the microorganisms or the cells dyed are not necessarily discolored, and not all fluorescent contaminants remain discolored.

If the fluorescent contaminants are discolored together with the microorganisms or the cells that are subjected to fluorescent labeling, measurement errors might be induced. It is therefore not possible to count the number of the microorganisms or the cells with high accuracy. Once the condition of the sample change, it is difficult to accurately count the number of the microorganisms or the cells.

Especially when the sample is liquid, the microorganisms or the cells and the contaminants are changed their locations upon fluorescent labeling, light irradiation and discoloration. For accurate counting, the overall sample to be detected should be monitored. In general, since a field of view of a microscope is small, a wide scan is needed and a long measurement time is unavoidably required. Especially when the overall sample is not observed, suction filtration is required before the sample is captured on a filter and then labeled with the fluorescent reagent. It is thus time consuming and cumbersome.

Patent Reference 1

Japanese Laid-Open Patent Application Publication No. 10-215894

The present invention is made in view of the above. Accordingly, it is an object of the present invention to provide a method of counting microorganisms or cells by eliminating the effects of fluorescent contaminants regardless of the form of a sample and preventing changes in the locations of the microorganisms or the cells and the contaminants in the sample so that the measurement accuracy can be improved and the measurement procedure can be simplified.

### DISCLOSURE OF THE INVENTION

In order to solve the above-mentioned problems, according to the present invention as claimed in claim 1, a method of counting microorganisms and/or tissue cells in a sample by labeling the microorganisms or the cells with a fluorescent labeling reagent, comprises the steps of:

1) contacting the sample containing the microorganisms or the cells with an adhesive sheet having an adhesive layer laminated on at least one surface of a substrate to capture the sample,

2) before fluorescent-labeling the microorganisms or the cells, obtaining a fluorescent image (a first image) of the sample, and then counting the number of luminous points in the first image,

3) after fluorescent-labeling the microorganisms or the cells, obtaining a fluorescent image (a second image) of the sample, and counting the number of luminous points in the second image,

4) counting the number of the microorganisms or the cells by a difference in the number of luminous points between the first and second images.

According to the counting method, the effects of fluorescent contaminants are eliminated regardless of the form of the sample, whereby the measurement accuracy can be improved. Additionally, the sample to be measured is adhered and fixed on the adhesive sheet, whereby changes in the locations of the microorganisms or the cells are prevented so that the measurement accuracy can be further improved and the measurement procedure can be simplified.

The term microorganisms herein means prokaryotes such as bacteria and actinomycetes, eucaryotes such as yeasts and moulds, lower algae and viruses. The cells means cultured cells derived from animals and plants, and pollens such as Japanese cryptomeria and Hinoki.

The term “physiologically active” means intracellular esterase or respiratory active. In the present invention, dyeing is performed using the fluorescent reagent that can emit fluorescence by the activation. As the result of the dyeing, the microorganisms or the cells labeled with the fluorescent reagent are referred to as “physiologically active microorganisms or the cells.”

The adhesive sheet has sufficient adhesive properties to capture the microorganisms or the cells on the sample to be detected, and includes an adhesive layer having a smooth surface structure laminated on a substrate.

The adhesive layer is not especially limited as long as it has sufficient adhesive properties to capture the microorganisms on the sample to be detected. Preferably, the adhesive layer includes a water insoluble adhesive agent, since a fluorescent material is not easily immersed into the adhesive layer, and the microorganisms or the cells captured as the adhesive layer is fused are not easily moved, when the microorganisms or the cells are labeled with the fluorescent reagent.

As the water insoluble adhesive, an acrylic adhesive, a rubber adhesive, and a silicone adhesive can be used. It is preferable that the acrylic adhesive and the silicone adhesive having good transparency and emitting less spontaneous fluorescence are used, from the viewpoint that they less affect the optical properties when obtaining the fluorescent image.

The acrylic adhesive comprises at least one alkyl (meth)acrylate as a main monomer and at least one hydrophilic monomer as a comonomer. The alkyl (meth)acrylate includes ethyl (meth)acrylate, propyl (meth)acrylate, butyl (meth)acrylate, hexyl (meth)acrylate, octyl (meth)acrylate, 2-ethylhexyl (meth)acrylate, nonyl (meth)acrylate and decyl (meth)acrylate. The hydrophilic monomer includes (meth)acrylic acid, itaconic acid, maleic acid, hydroxyethyl (meth)acrylate, methoxyethyl (meth)acrylate, ethoxyethyl (meth)acrylate, butoxyethyl (meth)acrylate and ethylene glycol (meth)acrylate.

The adhesive layer including the adhesive agent is preferably cross-linked by treating with a thermal cross-linking agent such as an isocyanate compound, an organic peroxide, an epoxy group containing compound and a metal chelate compound, or irradiating radioactive rays such as ultraviolet rays, γ rays and electron rays for better adhesive properties from the viewpoint of a shape retention.

The rubber adhesive comprises a main polymer and a tackifier incorporated therewith. Examples of the main polymer include natural rubber, polyisobutylene, polyisoprene, polybutene, a styrene-isoprene block copolymer and a styrene-butadiene block copolymer. Examples of the tackifier include a rosin resin, a terpene resin, a coumarone
indene resin, a terpene-phenol resin and a petroleum resin.

As the silicone adhesive, for example, the adhesive including dimethyl polysiloxane as a main component can be used.

Preferably, the adhesive layer has a thickness of 5 to 100 μm from the viewpoint of adhesion and follow-up properties to the surface to be detected, and capture of the microorganisms. When the fluorescent image of the microorganisms or the cells is obtained, smoothness (convexoconcave difference) on the surface of the adhesive layer is within a depth of focus of the image obtaining means, and preferably and practically 20 μm or less. When the smoothness is 20 μm or less, the fluorescent image obtaining means can match the focuses more precisely, whereby the image can be processed more accurately. The smoothness can be determined by observing the cross-section of the adhesive sheet using a surface roughness tester or an electron microscope, and measuring a mean height from the summit of a convex part to the lowest of a concave part on the surface of the adhesive agent.

Various materials can be used for the substrate of the adhesive sheet, as long as the materials do not form great convexoconcave on the surface of the adhesive layer and are flexible that can be freely applied and pressed to a curved surface or a narrow surface. Examples include polyester, polyethylene, polyurethane, polyvinyl chloride, woven fabric, non-woven fabric, paper and polyethylene laminated paper. Among them, polyester, polyethylene, polyvinyl chloride and polyurethane are preferably used since they have good smoothness. The substrate has a non-limiting thickness, only if it has sufficient strength as a supporting material, but preferably a thickness of about 5 to 200 μm.

The adhesive sheet can be formed by applying the adhesive layer including the above-mentioned adhesive agent to the above-mentioned substrate using the conventionally known method. Thus-obtained adhesive sheet can be cut into the desired shape and can be used.

In order to solve the above-mentioned problems, according to the present invention as claimed in claims 2 to 4, a method of counting microorganisms or cells according to claim 1, instead of the steps 2) to 4) comprises the steps of:

2) before fluorescent-labeling the microorganisms or the cells, obtaining a fluorescent image (a first image) of the sample,

3) after fluorescent-labeling the microorganisms or the cells, obtaining a fluorescent image (a second image) of the sample,

4) determining a differential image between the first and second images and determining the number of luminous points in the differential image to determine the number of the microorganisms or the cells (claim 2).

A method of counting microorganisms or cells according to claim 1, instead of the steps 2) to 4) comprises the steps of:

2) before fluorescent-labeling the microorganisms or the cells, obtaining a fluorescent image (a first image) of the sample, obtaining location information of luminous points in the first image, and setting radii, lengths or widths of non-sensitive regions to the luminous points in advance to recognize the non-sensitive regions attached to the individual luminous points in the first image,

3) after fluorescent-labeling the microorganisms or the cells, obtaining a fluorescent image (a second image) of the sample, and obtaining location information of luminous points in the second image,

4) determining the number of luminous points among the luminous points in the second image located outside the non-sensitive regions attached to the individual luminous points in the first image to determine the number of the microorganisms or the cells (claim 3).

The non-sensitive regions can be set so that “each region having a radius of 10 μm to the location of the luminous point obtained in the first image” or “each region having the image of +5 pixels in a length and a width”, depending on the counting status or the conditions of the sample. When the non-sensitive regions are adequately set, the number of the microorganisms and cells can be adequately counted.

A method of counting physiologically active microorganisms or the cells by labeling the physiologically active microorganisms or the cells with a fluorescent labeling reagent utilizes a metabolic action of the physiologically active microorganisms or the cells in the counting method according to any one of claims 1 to 3 (claim 4). As the reagent that are metabolized in organisms or cells and exhibit fluorescence, the above-mentioned FDA, CFDA, CTC and the like can be used.

A method of counting microorganisms or cells according to claim 4, wherein the physiologically active microorganisms or the cells are preferably bacteria (claim 5) in view of application advantages.

A method of counting microorganisms or cells according to any one of claims 1 to 4, wherein the second image is obtained by adding the fluorescent labeling reagent to the sample captured on the adhesive sheet, and then removing the fluorescent labeling agent that is not taken to the microorganisms or the cells with a cleaning liquid (claim 6).

### BRIEF DESCRIPTION OF THE DRAWINGS

FIG. 1 shows procedures for counting microorganisms or cells according to an embodiment of the present invention;

FIG. 2 shows schematic explanatory drawings of differential images according to an embodiment of the present invention;

FIG. 3 shows schematic explanatory drawings for location information of luminous points and;

FIG. 4 shows procedures for counting microorganisms or cells of a liquid sample according to an embodiment of the present invention.

### REFERENCE NUMERALS

1: solid sample, 2: adhesive sheet, 3: means for obtaining a fluorescent image, 4: image, 5: fluorescent labeling reagent, 16: cleaning liquid

### DETAILED DESCRIPTION OF THE PREFERRED EMBODIMENT

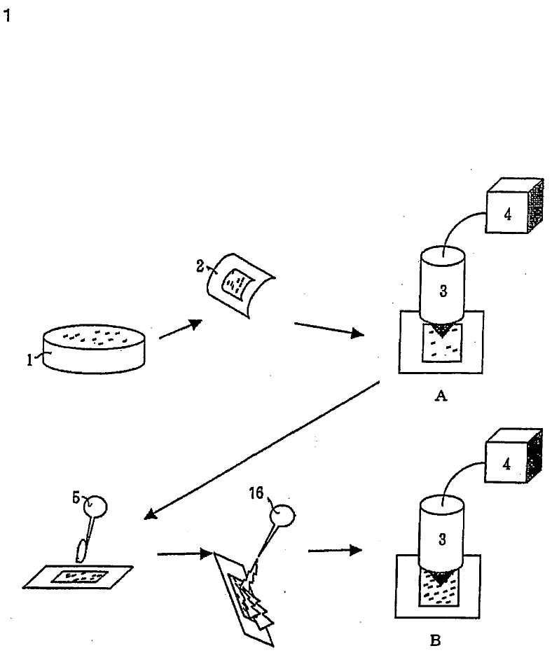

Embodiments of the present invention will be described below in detail referring to FIGS. 1 to 4.

### EXAMPLE 1

### Method of Counting by Determining a Difference in the Number of Luminous Points

Referring to FIG. 1, the embodiment mainly according to claims 1, 5 and 6 will be described below. This embodiment relates to a method of counting the number of bacteria contained in a solid sample by utilizing a difference between luminous points in the first and second images.

First, a solid sample 1 containing bacteria and contaminants is captured on the above-mentioned adhesive sheet 2. If the adhesive sheet is not used, the bacteria are generally captured by wiping or stomaching to deploy them into sterilizaed water. The use of the adhesive sheet simplifies the sampling operation.

Then, a means for obtaining a fluorescent image 3 is used to obtain a fluorescent image (first image) of the sample on the adhesive sheet containing the bacteria. The obtained image is image-processed at an image and arithmetic processing unit 4 to determine fluorescent luminous points A in the image. The luminous points already existed before luminescence labeling represent the contaminants.

Specifically, the image processing is performed as follows:
(1) A plurality of images are taken at the same field of view, and are averaged to decrease random noises.
(2) Shading (gradation) is corrected.
(3) Luminous points are extracted by an edge detection.
(4) A series of discrete luminous points are recognized by labeling.
(5) The luminous points within the region set in advance are selected.
(6) The luminous points selected in the step (5) are counted.

The region set in advance in the step (5) is determined by the size of the bacteria to be counted and by the features of the apparatus used for detection. By performing an experimental review in advance, “the region, for example, corresponding to a diameter of 0.2 to 10 μm” can be set.

Then, a fluorescent labeling reagent 5 is applied over the adhesive sheet. When DAPI or AO that has affinity for genes is used as the fluorescent labeling reagent 5, all bacteria can be labeled. Propidium iodide (PI) is suitable for labeling dead bacteria. When CFDA or CTC that develops fluorescence by biologically activity of the bacteria such as an enzyme reaction or breath is used, only viable bacteria can be selectively labeled. An antigen-antibody reaction or a technique of recognizing a specific gene arrangement is utilized to label the specific bacteria. In the former case, an antibody that is specifically bonded to an antigen of the bacteria to be detected is fluorescence labeled in advance, and the fluorescence labeled antibody is reacted with the sample to label only the specific bacteria. In the latter case, FISH or In situ PCR techniques is used for specific genes as a target to label the intended bacteria.

The fluorescent labeling reagent that is not reacted with the bacteria may produce a noise of the measurement. In this case, the reagent that is not taken into the bacteria is effectively removed by a cleaning liquid 16. The bacteria and fluorescent contaminants are adhered and fixed and therefore do not flow, resulting in an errorless measurement.

Preferably, the cleaning liquid is a buffer solution having a composition and a pH suitable for developing fluorescent labeling. For example, AO or CFDA can develop effectively fluorescence within neutral to alkalinity ranges. The buffer solution having such a pH is used to provide a high contrast fluorescent image. Specifically, the pH is preferably 6.5 to 9.0. In view of decreasing non-specific dyeing, the pH is more preferably 7.0 to 8.5, still more preferably 7.0 to 8.0.

The buffer liquid desirably contains a stable buffer component at the pH as defined above. Specific examples include a phosphate, a borate and a tris salt. Especially preferable is a phosphate. The concentration of the buffer component may be suitably selected depending on its type. From a stable dyeing, the concentration of the buffer component is preferably 1 to 500 mM, and more preferably 5 to 300 mM. The buffer liquid may contain sodium chloride or sugars in order to maintain isotonicity with the microorganisms or the cells.

After the fluorescent labeling, an fluorescent image (second image) is again obtained of the sample on the adhesive sheet. The obtained second image is image-processed similar to the first image to determine fluorescent luminous points B in the image. Then, the difference (B-A) in the number of luminous points between the first and second images, i.e., the number of luminous points appeared by the fluorescent labeling is determined to obtain the number of the bacteria.

### EXAMPLE 2

### Method of Counting by Utilizing a Differential Image

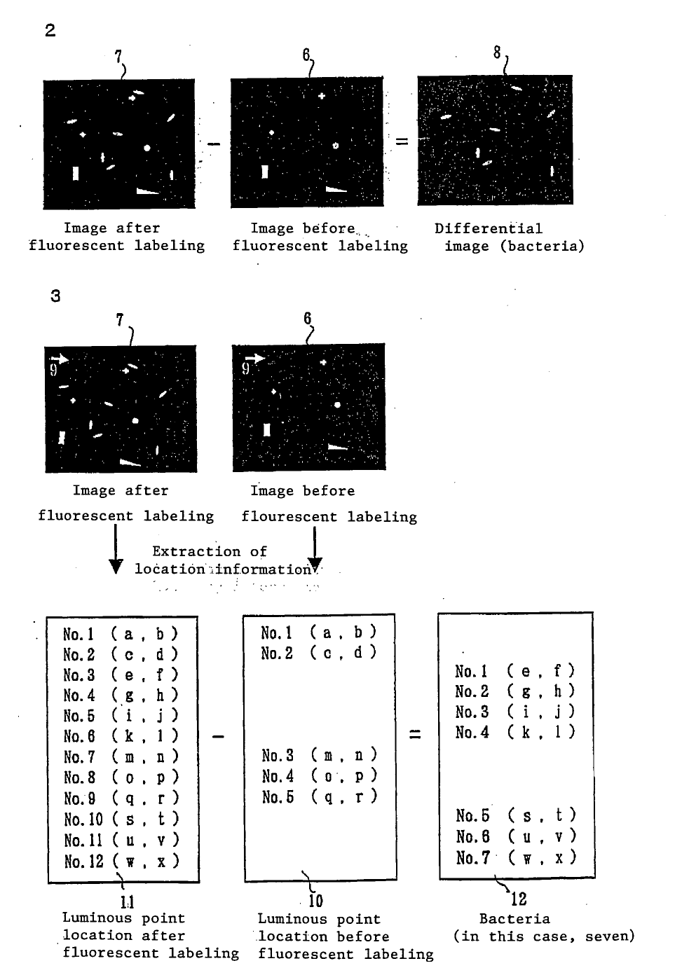

Referring to FIG. 2, the embodiment according to claim 2 will be described below. This embodiment relates to a method of counting the number of bacteria by utilizing a differential image between the first and second images.

First, a sample containing the bacteria is captured on the above-mentioned adhesive sheet similar to embodiment 1. Next, a fluorescent image of the sample containing the bacteria (a first image 6 shown in a central part of FIG. 2) on the adhesive sheet is obtained. Then, a fluorescent labeling reagent is applied over the adhesive sheet to fluorescent-label the bacteria. After the fluorescent labeling reagent that is not taken to the bacteria is cleaned with a cleaning liquid, a fluorescent image of the sample (a second image 7 shown in a left part of FIG. 2) on the adhesive sheet is obtained. A differential image 8 shown in a right part of FIG. 2 is obtained from the second image 7 and the first image 6.

The luminous points existing in the differential image 8 appear by the luminescence labeling, and are counted as the number of the bacteria. As described above, when the bacteria were not held with the adhesive sheet, the luminous points containing the bacteria were moved by the fluorescent labeling and the cleaning. Therefore, it was difficult to provide an ideal differential image. In order to prevent the bacteria from moving, the bacteria were subjected to the fluorescent labeling and the cleaning, while they were suction-filtrated. However, this example can solve the problems and can count the number of the bacteria with a simplified operation.

The ideal results may not be obtained with the practical differential image using the above-described processing. Specifically, it is difficult to obtain the accurate number of the luminous points in the simple differential image in the following cases:
(1) If the image obtaining locations are mismatched between the first and second images:
(2) If the luminances of the first and second images are different as a whole (background) by the effect of the fluorescent labeling operation and the optical conditions when the image is obtained;
(3) If respective luminous points of the first image are different from the corresponding respective luminous points of the second image in the shapes or sizes due to the difference of focusing when the image is obtained, or the difference of setting the conditions when the image is processed; and
(4) If the luminous points are moved in the first and second images.

In the case (1), a pattern matching technique is used to recognize the location relationship between two images, and to determine a differential image of the corresponding location, thereby attaining a correct result.

In the case (2), using an image processing such as a binalizing or edge detection technique, a signal derived from the luminous points is distinguished from a signal derived from a background. In the case (3), this example is less suitable, but Example 3 described later can handle it effectively. In the case (4), a difference between the images is difficult to be adjusted, but Example 1 described above can handle it.

### EXAMPLE 3

### Method of Counting by Utilizing Location Information

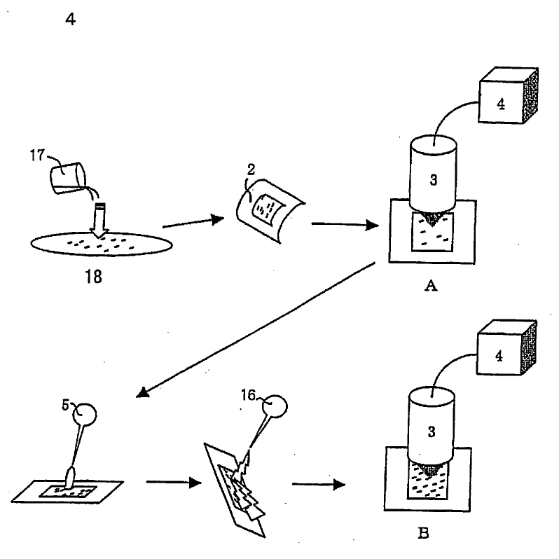

Referring to FIG. 3, the embodiment according to claim 3 will be described below. This embodiment relates to a method of counting the number of bacteria by utilizing a location information of luminous points.

Similar to Examples 1 and 2, the sample containing the bacteria is captured on the above-mentioned adhesive sheet. Then, a fluorescent image (first image 6) of the sample containing the bacteria on the adhesive sheet is obtained. When the image is obtained, a reference point 9 (shown in the drawing as a white arrow) is used to find the location of the sample on the adhesive sheet and to recognize the location information 10 of the luminous points in the first image. A fluorescent labeling reagent is applied over the adhesive sheet to fluorescent-labeling the bacteria. Thereafter, a fluorescent image (second image 7) of the sample containing the bacteria on the adhesive sheet is obtained. Similar to the first image, when the image is obtained, the reference point 9 (shown in the drawing as an arrow) is used to find the location of the sample on the adhesive sheet and to recognize the location information 11 of the luminous points in the second image.

In the first image 6 and the second image 7 shown in FIG. 3, a parallel displacement and a rotation slightly occur. Thus, if the image obtaining locations are mismatched between the first and second images, the locations of the luminous points can be accurately recognized as the corresponding locations from the reference point. The locations of the luminous points in the second image 7 and the first image 6 are compared to determine luminous point information 12 that newly appears when the second image 7 is obtained. The numbers of the luminous points determined by the luminous point information 12 are the numbers of the bacteria.

In practice, it is difficult to determine match or mismatch of the points having no surface areas. Accordingly, predetermined regions set in advance are recognized as non-sensitive regions attached to the individual luminous points to the locations of the luminous points obtained in the first image 6. As described above, the non-sensitive regions are set so that “each region having a radius of 10 μm to the location of the luminous point obtained in the first image” or “each region having the image of ±5 pixels in a length and a width.” The non-sensitive regions are adequately set, whereby the number of the bacteria can be adequately counted in the case of (3) that is not attained in Example 2 as described above.

### EXAMPLE 4

### Liquid Sample

Referring to FIG. 4, a method of counting the number of bacteria contained in the liquid sample will be described below.

Firstly, a liquid sample 17 containing bacteria is filtrated and captured on a filter 18. The filter 18 is preferably a membrane filter having a highly uniform pore size. The pore size of the membrane filter should be selected depending on the size of the bacteria to be counted, and is generally about 0.2 to 0.6 μm for counting the number of the bacteria.

Then, the sample containing the bacteria and the contaminants captured on the filter 18 are captured on the adhesive sheet 2. The subsequent operations are the same as in Example 1, and the description thereof are not repeated.

### INDUSTRIAL APPLICABILITY

According to the present invention, the reagent is applied to the microorganisms or tissue cells to emit fluorescence, as described above, and the fluorescent images are utilized to count the number of the microorganisms or the cells in the sample. The microorganisms include prokaryotes such as bacteria and actinomycetes, eucaryotes such as yeasts and moulds, algae and viruses. The tissue cells include cultured cells derived from animals and plants, and pollens such as Japanese cryptomeria and Hinoki. The counting method of the present invention is applied to medical, food manufacturing, and city water or sewage water fields.

According to the present invention, a method of counting microorganisms or cells in a sample by labeling the microorganisms or the cells with a fluorescent labeling reagent, which comprises contacting the sample containing the microorganisms or the cells with an adhesive sheet having an adhesive layer laminated on at least one surface of a substrate to capture the sample; before fluorescent-labeling the microorganisms or the cells, obtaining a fluorescent image (a first image) of the sample; after fluorescent-labeling the microorganisms or the cells, obtaining a fluorescent image (a second image) of the sample, and then determining the difference in the number of luminous points between the first and second images, or determining a differential image between the first and second images and determining the number of luminous points in this differential image, or determining the number of luminous points among the luminous points in the second image located outside non-sensitive regions attached to the individual luminous points in the first image.

According to the method of counting the microorganisms or the cells, the effects of fluorescent contaminants are eliminated regardless of the form of the sample and changes in the locations of the microorganisms or the cells and the contaminants in the sample are prevented so that the measurement accuracy can be improved and the measurement procedure can be simplified.

1. A method of counting microorganisms or cells in a sample by labeling the microorganisms or the cells with a fluorescent labeling reagent, comprising the steps of:
1) contacting the sample containing the microorganisms or the cells with an adhesive sheet having an adhesive layer laminated on at least one surface of a substrate to capture the sample,
2) before fluorescent-labeling the microorganisms or the cells, obtaining a fluorescent image (a first image) of the sample, and then counting the number of luminous points in the first image,
3) after fluorescent-labeling the microorganisms or the cells, obtaining a fluorescent image (a second image) of the sample, and counting the number of luminous points in the second image,
4) counting the number of the microorganisms or the cells by a difference in the number of luminous points between the first and second images.

2. A method of counting microorganisms or cells according to claim 1, instead of the steps 2) to 4) comprising the steps of:
2) before fluorescent-labeling the microorganisms or the cells, obtaining a fluorescent image (a first image) of the sample,
3) after fluorescent-labeling the microorganisms or the cells, obtaining a fluorescent image (a second image) of the sample,
4) determining a differential image between the first and second images and determining the number of luminous points in the differential image to determine the number of the microorganisms or the cells.

3. A method of counting microorganisms or cells according to claim 1, instead of the steps 2) to 4) comprising the steps of:
2) before fluorescent-labeling the microorganisms or the cells, obtaining a fluorescent image (a first image) of the sample, obtaining location information of luminous points in the first image, and setting radii, length or width of non-sensitive regions to the luminescence spots in advance to recognize the non-sensitive regions attached to the individual luminous points in the first image,
3) after fluorescent-labeling the microorganisms or the cells, obtaining a fluorescent image (a second image) of the sample, and obtaining location information of luminous points in the second image,
4) determining the number of luminous points among the luminous points in the second image located outside the non-sensitive regions attached to the individual luminous points in the first image to determine the number of the microorganisms or the cells.

4. A method of counting physiologically active microorganisms or the cells by labeling the physiologically active microorganisms or the cells with a fluorescent labeling reagent utilizing a metabolic action of the physiologically active microorganisms or the cells in the counting method according to claim 1.

5. A method of counting microorganisms or cells according to claim 4, wherein the physiologically active microorganisms or the cells are bacteria.

6. A method of counting microorganisms or cells according to claim 1, wherein the second image is obtained by adding the fluorescent labeling reagent to the sample captured on the adhesive sheet, and then removing the fluorescent labeling agent that is not taken to the microorganisms or the cells with a cleaning liquid.
